(19) United States
(12) Patent Application Publication
(10) Pub. No.: US 2007/0231901 A1
(43) Pub. Date: Oct. 4, 2007

(54) Microfluidic cell culture media

(76) Inventors: Shuichi Takayama, Ann Arbor, MI (US); Nobuyuki Futai, Ann Arbor, MI (US); Yun Seok Heo, Ann Arobor, MI (US); Gary Daniel Smith, Ann Arbor, MI (US); Jonathan Wanserk Song, Ann Arbor, MI (US)

Correspondence Address: BROOKS KUSHMAN P.C., 1000 TOWN CENTER, TWENTY-SECOND FLOOR, SOUTHFIELD, MI, 48075, US

(21) Appl. No.: 11/607,388

(22) Filed: Dec. 1, 2006

Related U.S. Application Data
(60) Provisional application No. 60/741,864, filed on Dec. 2, 2005.
(60) Provisional application No. 60/741,665, filed on Dec. 2, 2005.

(51) Int. Cl. C12N 1/20; C12N 5/00

(52) U.S. Cl. 435/404; 435/253.6

(57) ABSTRACT

A microfluidic cell culture media may have an initial concentration of an active material less than a therapeutic window of concentration necessary to provide a cellular mass a therapeutic environment.

This application claims the benefit of the following U.S. provisional applications: 60/741,864 filed Dec. 2, 2005; 60/741,665 filed Dec. 2, 2005.

### STATEMENT REGARDING FEDERALLY SPONSORED RESEARCH OR DEVELOPMENT

This invention was made with Government support under at least one of: NASA award NNC04AA21A; Army Research Office award DAAD19-03-1-0168; and, National Science Foundation Award BES0238265 The Government has certain rights to the invention.

### BACKGROUND

1. Field of the Invention

The invention relates to microfluidic cell culture media.

2. Discussion

Microfluidic devices allow a user to work with nano- to microliter volumes of fluids and are useful for reducing reagent consumption, creating physiologic cell culture environments that better match the fluid-to-cell-volume ratios in vivo, and performing experiments that take advantage of low Reynolds number phenomenon such as subcellular treatment of cells with multiple laminar streams. Many microfluidic systems are made of polydimethylsiloxane (PDMS) because of its favorable mechanical properties, optical transparency, and bio-compatibility.

### SUMMARY

Embodiments of the invention may take the form of a microfluidic cell culture media for a cellular mass in a microfluidic cell culture device. The media provides the cellular mass a therapeutic environment. The cellular mass has an active material. The media includes a fluid having an initial concentration of the active material. The active material of the fluid is capable of being used by the cellular mass for at least a portion of one cell process. The fluid is capable of receiving active material released by the cellular mass. The initial concentration is less than a therapeutic window of concentration necessary to provide the cellular mass a therapeutic environment.

Embodiments of the invention may take the form of a microfluidic cell culture media for use, during a cell culture period, with a cellular mass in a microfluidic cell culture device. The device is configured to at least one of absorb a portion of the media and permit a portion of the media to evaporate. The media includes a water-based fluid having an initial concentration of an active material less than a therapeutic window of concentration necessary to provide the cellular mass a therapeutic environment during the cell culture period. The initial concentration has a value such that if a predetermined amount of water from the fluid is at least one of absorbed and evaporated during the cell culture period, the concentration of the active material adjacent the cellular mass will fall within the therapeutic window of concentration.

Embodiments of the invention may take the form of a microfluidic cell culture media for use, during a cell culture period, with a cellular mass in a microfluidic cell culture device. The device is configured to at least one of absorb a portion of the media and permit a portion of the media to evaporate. The media includes a water-based fluid having an initial osmolality less than a therapeutic window of osmolality necessary to provide the cellular mass a therapeutic environment during the cell culture period. The initial osmolality has a value such that if a predetermined amount of water from the fluid is at least one of absorbed and evaporated during the cell culture period, the osmolality of the fluid adjacent the cellular mass will fall within the therapeutic window of osmolality.

### BRIEF DESCRIPTION OF THE DRAWINGS

FIG. 1 is an exploded, perspective view of a microfluidic cell culture device;

FIG. 2a is a top view of a substrate of the system of FIG. 1;

FIG. 2b is a side view, and in cross-section, of the substrate taken along section line 2b-2b in FIG. 2a;

FIG. 2c is a bottom view of the substrate of FIG. 2a;

FIG. 3a is a side view, partially broken-away and in cross-section, of a membrane of the system of FIG. 1 with a pair of actuator pins in engagement with a lower surface of the membrane;

FIG. 3b is a side view, partially broken-away and in cross-section, of an alternative embodiment of a membrane of the system of FIG. 1 with a pair of actuator pins spaced away from the lower surface of the membrane;

FIG. 4a is a side view, partially broken-away and in cross-section, of an alternative embodiment of the substrate of FIG. 1 and the membrane of FIG. 3b and illustrating two different heights of a biological fluid in a pair of reservoirs formed in the substrate;

FIG. 4b is an enlarged side view, partially broken-away and in cross-section, of the substrate and membrane of FIG. 4a and illustrating the relative heights of a cell mass and an end portion of a passageway;

FIG. 4c is another enlarged side view, partially broken-away and in cross-section, of the substrate and membrane of FIG. 4a and illustrating the angle of the surface which defines a reservoir and the relative widths of a cell mass and a lower portion of the reservoir;

FIG. 5a is an enlarged side view, partially broken-away and in cross-section, of an alternative embodiment of a substrate and membrane of FIG. 1 and illustrating a cell mass retained in a lower portion of the reservoir above the passageway;

FIG. 5b is another enlarged side view, partially broken-away and in cross-section, of an alternative embodiment of a substrate and membrane of FIG. 1 and illustrating a cell mass retained in a lower portion of the reservoir below the passageway; and

FIG. 5c is yet another enlarged side view, partially broken-away and in cross-section, of an alternative embodiment of a substrate and membrane of FIG. 1 and illustrating a cell mass with a width smaller than the width of the lower portion of the reservoir.

### DETAILED DESCRIPTION

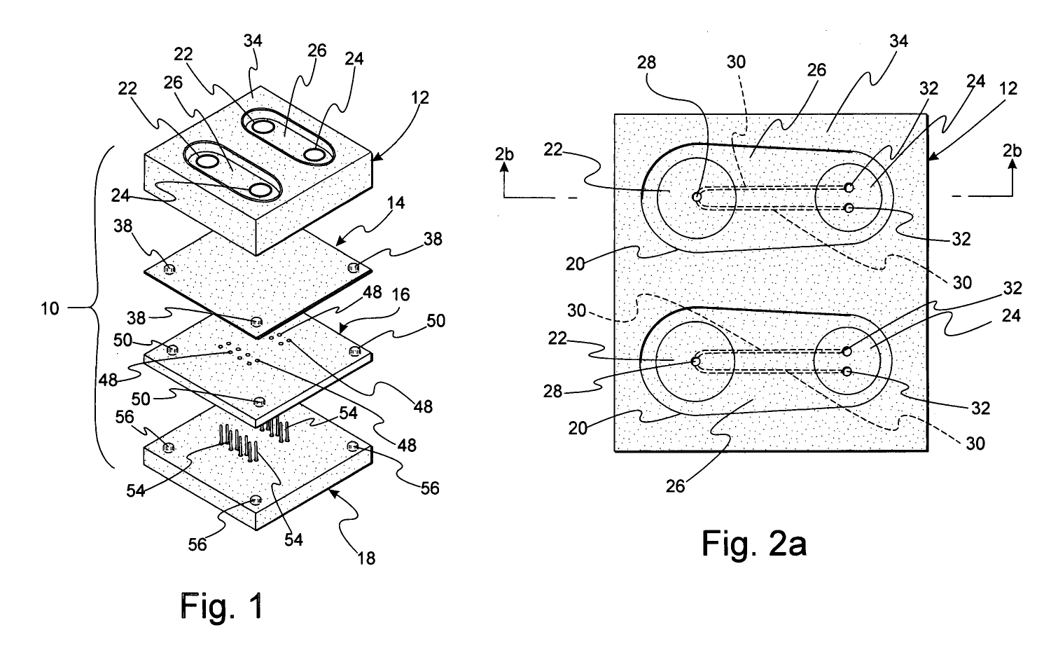

FIG. 1 is an exploded, perspective view of microfluidic cell culture system or device 10. Device 10 includes substrate 12 configured to receive a cellular mass, e.g., an embryo, as explained in detail below, non-rigid membrane 14, locating block 16, and pin actuating device 18.

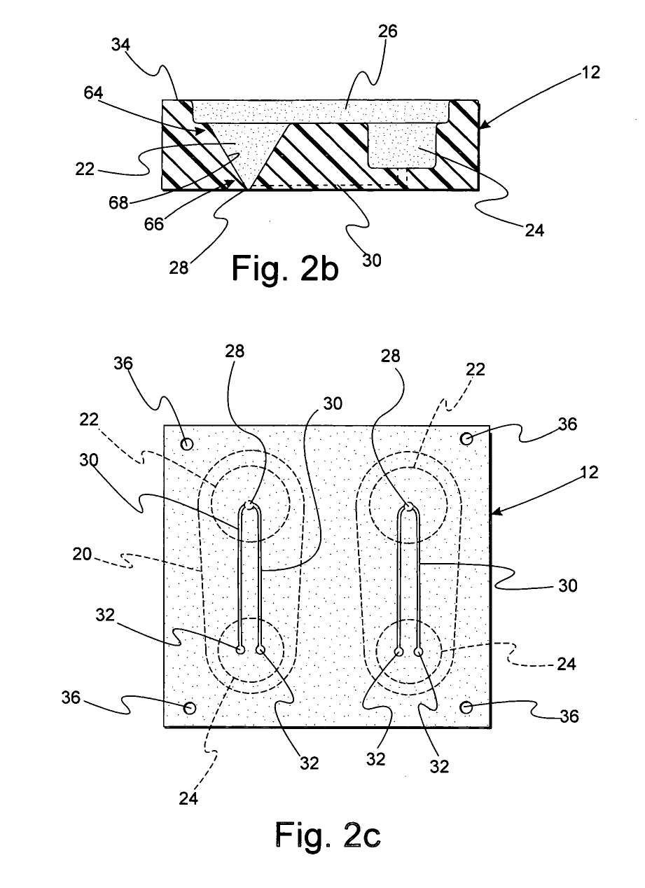

FIG. 2a is a top view of substrate 12. Substrate 12 includes funnel 22, reservoir 24, and overlay reservoir 26. Bottom portion 28 of funnel 22 is in fluid communication with reservoir 24 via microchannel 30. Microchannel 30 has a volume less than 1 microliter. Reservoir 24 includes reservoir openings 32 which provide openings to microchannel 30 such that fluids may travel between funnel 22 and reservoir 24 as explained in detail below.

FIG. 2b is a side view, and in cross-section, of substrate 12 taken along section line 2b-2b in FIG. 2a. A portion of microchannel 30 is formed in substrate 12 while another portion of microchannel 30 is formed by membrane 14 as described in detail below. Microchannel 30, however, may be completely formed in substrate 12 or in any other suitable fashion. Microchannel 30 may have a square, circular, bell, or any other suitably shaped cross-section. Substrate 12 further includes hydrophilic surface 34 to promote fluid retention within overlay reservoir 26.

Fluid may move between funnel 22 and reservoir 24 via localized deformation of membrane 14. Fluid may also move between funnel 22 and reservoir 24 under the influence of gravity as explained in detail below.

Substrate 12 may be optically transparent and made from such materials as plastic, e.g., PDMS, polymethylmethacrylate, polyurethane, or glass.

FIG. 2c is a bottom view of substrate 12. Substrate 12 includes female locators 36 which assist in locating substrate 12 relative to membrane 14 as explained in detail below.

Substrate 12 may comprise a thick, e.g., 8 mm, PDMS slab, fabricated by using soft lithography. The PDMS slab may be prepared by casting a prepolymer (Sylgard 184, Dow-Corning) at a 1:10 curing agent-to-base ratio against positive relief features. Relief features may comprise SU-8 (MicroChem, Newton, Mass.) and be fabricated on a thin, e.g., 200 μm, glass wafer by using backside diffused-light photolithography. The prepolymer may then cure at 60° C. for 60 minutes, and holes may be punched by a sharpened 14-gauge blunt needle.

Substrate 12 may comprise two layers of cured PDMS at a ratio of 1:10 base to curing agent sealed together irreversibly using plasma oxidation (SPI supplies, West Chester, Pa.). Funnel 22 and reservoir 24 are formed in the top layer. Microchannel 30 is formed in the bottom layer using soft lithography. Microchannel 30 faces downward and may be sealed against membrane 14 as explained in detail below.

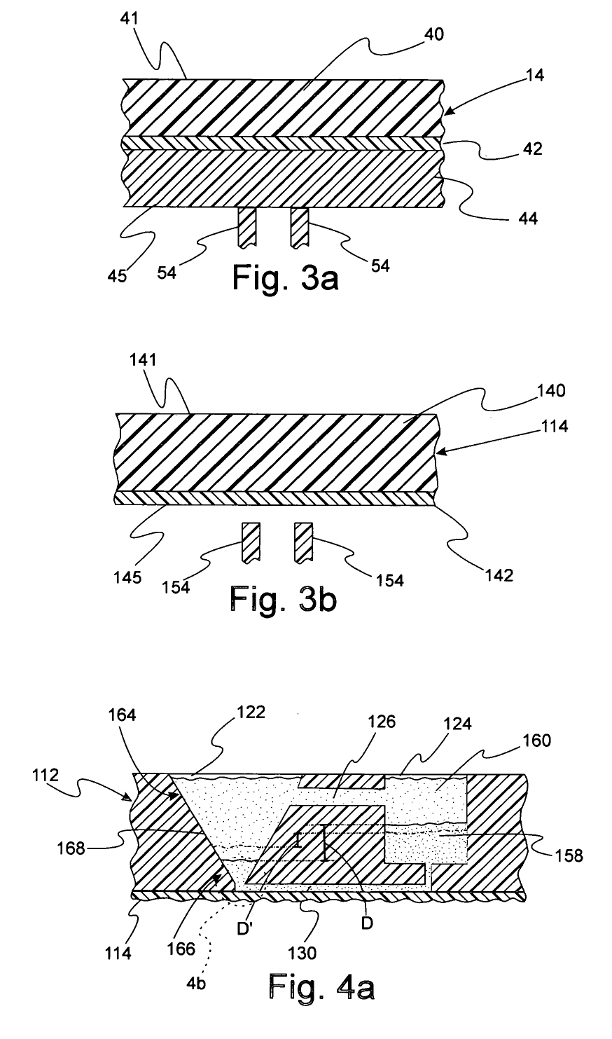

FIG. 3a is a side view, partially broken-away and in cross-section, of membrane 14 and pins 54 of pin actuating device 18 (FIG. 1). Membrane 14 includes male locators 38 (FIG. 1) configured to be received by female locators 36 of substrate 12 to locate membrane 14 relative to substrate 12.

Membrane 14 is optically transparent and includes top layer 40, upper surface 41, middle layer 42, bottom layer 44, and bottom surface 45. Top layer 40 and bottom layer 42 comprise PDMS. Middle layer 42 comprises parylene. Top layer 40 and bottom layer 44, alternatively, may comprise any suitable non-rigid, bio-compatible polymer such as a non-rigid plastic, e.g., polyurethane, or a hyrdrogel, e.g., polyvinylalcohol. Middle layer 42, alternatively, may comprise any suitable non-rigid polymer such as polyvinylidene chloride or polyurethane.

Top layer 40 and bottom layer 44 may have a combined thickness of less than 1 mm, e.g., 200 μm. Middle layer 42 may range in thickness from 2-20 μm, e.g., 2-5 μm.

Pins 54 of pin actuating device 18 may selectively extend from the position shown into membrane 14 to locally deform membrane 14 such that at least a portion of top layer 40 extends into microchannel 30 (FIG. 2b). The selective actuation of pins 54 may move a fluid in microchannel 30 or prevent, or impede, the movement of the fluid in microchannel 30 as explained in detail below.

Middle layer 42 minimizes evaporation of a fluid, e.g., a water based fluid, contained within microchannel 30 to prevent, for example, undesirable shifts in osmolality of the fluid. Middle layer 42 is also resistant to the flow of at least one gas, such as oxygen and carbon dioxide, from microchannel 30 and provides mechanical durability and stability against cracking caused by the selective actuation of pins 54. Fatigue from the actuation of pins 54 does not substantially increase middle layer's 42 ability to substantially reduce the rate at which a fluid from microchannel 30 moves through membrane 14.

Membrane 14 includes female locators (not shown) which are used to locate membrane 14 relative to locating block 16 as explained in detail below.

Membrane 14 may be prepared by spin-coating PDMS onto a 4″ silanized silicon wafer to a thickness of 100 μm, curing this layer at 120° C. for 30 minutes, depositing a 2.5 or 5 μm thick parylene layer, plasma oxidizing the resulting parylene surface for 90 seconds, spin-coating another 100 μm thick layer of PDMS, and curing for a total thickness of approximately 200 μm.

FIG. 3b is a side view, partially broken-away and in cross-section, of an alternative embodiment of membrane 114 and pins 154 of pin actuating device 118 (not shown). Membrane 114 includes top layer 140, upper surface 141, bottom layer 142, and lower surface 145. Top layer 140 comprises PDMS and bottom layer 142 comprises polyvinylidene chloride. Top layer 140, alternatively, may comprise any suitable non-rigid, bio-compatible polymer such as a non-rigid plastic, e.g., polyurethane, or a hyrdrogel, e.g., polyvinylalcohol, whereas bottom layer 142 may comprise any suitable non-rigid polymer such as polyurethane.

Top layer 140 and bottom layer 142 may have a combined thickness of less than 1 mm, e.g., 200 μm.

Bottom layer 142 minimizes evaporation of a fluid, e.g., a water based fluid, contained within microchannel 30 to prevent, for example, undesirable shifts in osmolality of the fluid. Bottom layer 142 is also resistant to the flow of at least one gas, such as oxygen and carbon dioxide, from microchannel 30 and provides mechanical durability and stability against cracking caused by the selective actuation of pins 154. Fatigue from the actuation of pins 154 does not substantially increase bottom layer's 142 ability to substantially reduce the rate at which a fluid from microchannel 30 moves through membrane 114.

Membrane 114 may be prepared by spin-coating freshly mixed 1:10 PDMS onto silanized glass slides (Corning Glass Works, Corning, N.Y.) to a uniform thickness of either approximately 120 μm or 400 μm, curing overnight at 120° C., and then adhering polyvinylidene chloride via conformal contact with the PDMS.

Referring to FIG. 1, locating block 16 includes pin holes 48 and male locators 50. Pin holes 48 are configured to receive pins 54 of pin actuating device 18. Male locators 50 are configured to be received by the female locators of membrane 14 to locate locating block 16 relative to membrane 14. In particular, by locating block 16 relative to membrane 14, pin holes 48 are aligned with microchannel 30. Locating block 16 includes female locators (not shown) which are used to locate locating block 16 relative to pin actuating device 18 as explained in detail below.

Locating block 16 is rigid and optically transparent and made from such materials as polystyrene, cyclic olefin copolymer, glass, or metal.

Pin actuating device 18 is a Braille-type actuator as described in detail below. Pins 54 are actuated with a force of 18 g. Pins 54, however, may be actuated with a force ranging from approximate 3 g to 300 g. Pins 54 may be actuated, for example, 10 times per second or once a minute. Pins 54 may be actuated for a period ranging from minutes to weeks. Any suitable tactile device, however, may be used.

Pins 54 of pin actuating device 18, when actuated, extend and press upon membrane 14, restricting or closing microchannel 30. Pins 54 may be actuated in any suitable fashion such that a fluid flows between funnel 22 and reservoir 24 via microchannel 30. Pins 54 may also be actuated such that the fluid does not move between funnel 22 and reservoir 24 via microchannel 30.

Pin actuating device 18 includes male locators 56. Male locators 56 are configured to be received by female locators 52 of locating block 16 to align locating block 16 relative to pin actuating device 18. By aligning locators 46, 56, pins 54 are aligned with pin holes 48.

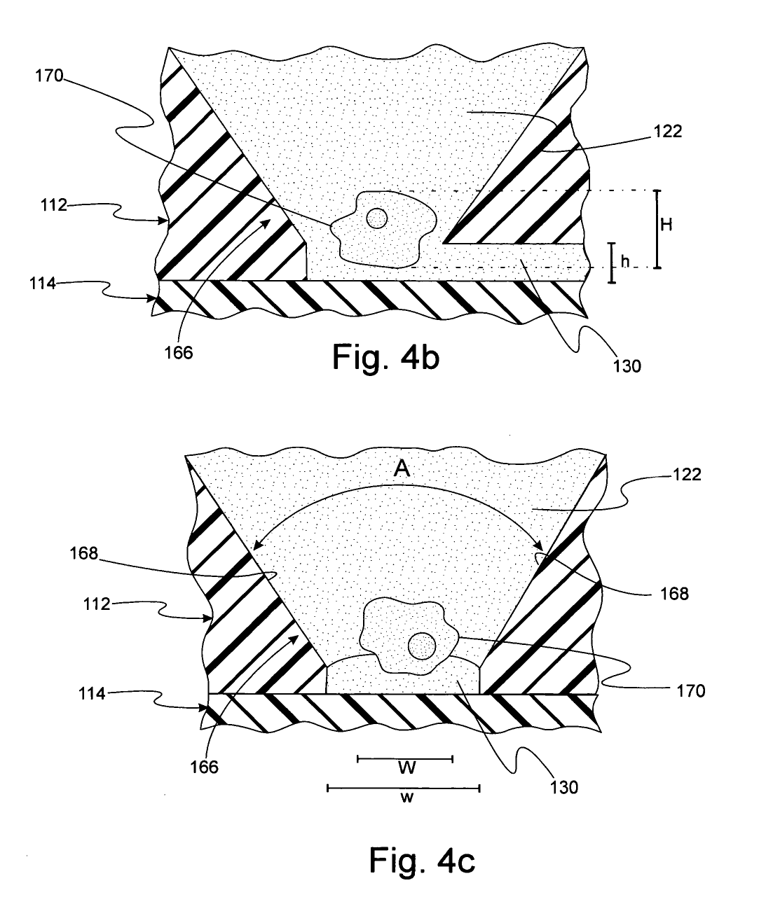

FIG. 4a is side view, partially broken-away and in cross-section, of substrate 112 and membrane 114. Reservoir 124 and funnel 122 are in fluid communication via microchannel 130. Bio-compatible fluid 158 may be transported between reservoir 124 and funnel 122 via localized deformation of membrane 114 by pin actuating device 118. D is the difference in height between bio-compatible fluid 158 in reservoir 124 and funnel 122.

Funnel 122 and reservoir 124 are further in fluid communication via upper channel 126. Microchannel 130 has a resistance to fluid flow greater than upper channel 126. Upper channel 126 is defined by a hydrophobic surface to, for example, repel bio-compatible fluid 158.

Immiscible fluid 160, e.g., an oil having a density lower than bio-compatible fluid 158, may move between funnel 122 and reservoir 124 via channel 126. Immiscible fluid 160 reduces evaporation of bio-compatible fluid 158 and reduces the flow of oxygen and carbon dioxide into and out bio-compatible fluid 158. Gravity will act upon immiscible fluid 160 such that the height of immiscible fluid 160 in funnel 122 will equal the height of immiscible fluid 160 in reservoir 124 thereby maintaining the difference in height, D, of bio-compatible fluid 158.

D′ is the desired difference in height between bio-compatible fluid 158 in funnel 122 and bio-compatible fluid 158 in reservoir 124 after pin actuating device 118, for example, has been used to move bio-compatible fluid 158 from reservoir 124 to funnel 122. Such a height may provide a desired amount of fluid in funnel 122 conducive to cell culturing. As bio-compatible fluid 158 is moved from reservoir 124 to funnel 122, immiscible fluid 160 will flow from funnel 122 to reservoir 124 via channel 126 under the influence of gravity such that in the absence of deformation of membrane 114 that would cause, for example, bio-compatible fluid 158 to further move between funnel 122 and reservoir 124 or prevent bio-compatible fluid 158 from moving between funnel 122 and reservoir 124, immiscible fluid 160 will substantially maintain the difference in height D′ under the influence of gravity for a desired period of time, e.g., approximately 30 minutes. Microchannel 130 and and channel 126 thus from a continuous fluid path between funnel 122 and reservoir 124.

Fluid may move between funnel 122 and reservoir 124 in any number of ways. For example, a pump may pump immiscible fluid 160 from one of funnel 122 and reservoir 124 to the other of funnel 122 and reservoir 124 thereby changing the height of bio-compatible fluid 158.

Funnel 122 includes upper portion 164 and lower portion 166. Surface 168 of funnel 122 tapers inwardly from upper portion 164 to lower portion 166. Furthermore, upper portion 164 has a width greater than lower portion 166.

The shape of funnel 122 facilities the one-step loading and unloading of cells into and out of lower portion 166. A pipette holding cells may be inserted into funnel 122 at an angle such that a user has a substantially unobstructed view of lower portion 166. Likewise, a pipette may be inserted into funnel 122 to remove cells from lower portion 166 such that a user has a substantially unobstructed view of lower portion 166.

FIG. 4b is an enlarged side view, partially broken-away and in cross-section, of funnel 122 and microchannel 130. Lower portion 166 of funnel 122 is configured to receive cellular mass 170. Cellular mass 170 has a cellular height H and microchannel 130 has a channel height h. Cellular mass 170 may be, for example, a human zygote, a mammalian zygote, a clump of mammalian cells, or a single mammalian cell. Microchannel 130 is configured such that cellular mass 170 will not exit lower portion 166 of funnel 122.

FIG. 4c is another enlarged side view, partially broken-away and in cross-section, of funnel 122 and microchannel 130 looking down the length of microchannel 130. Cellular mass 170 has a cellular width W and microchannel 130 has a channel width w. Cellular mass 170 also has a cellular length (not shown). Microchannel 130 may be configured such that at least one of the channel height h and the channel width w is less than at least one of the cellular height H, the cellular width W, and the cellular length L.

Angle A is defined by opposite surfaces 168 of funnel 122. Angle A may range between 30° and 160° inclusive.

At least one of the channel height h and the channel width w may be less than 250 μm or the width of human hair. In the case where cellular mass 170 is a denuded human zygote, at least one of the channel height h and the channel width w may be less than 140 μm. In the case where cellular mass 170 is a denuded mammalian zygote, at least one of the channel height h and the channel width w may be less than 70 μm. In the case where cellular mass 170 is a clump of mammalian cells, at least one of the channel height h and the channel width w may be less than 50 μm. In the case where cellular mass 170 is a single mammalian cell, at least one of the channel height h and the channel width w may be less than 5 μm.

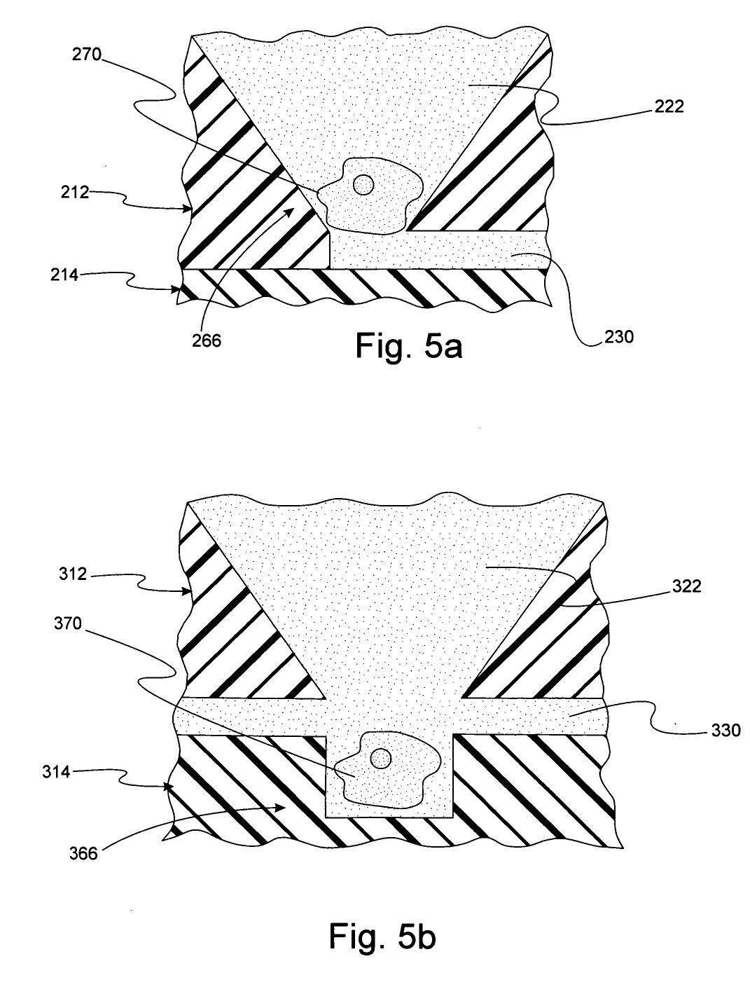

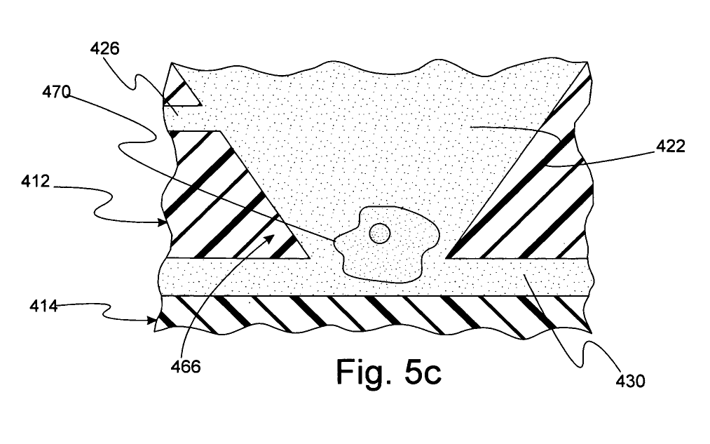

FIG. 5a is an enlarged side view, partially broken-away and in cross-section, of funnel 222 and microchannel 230. Lower portion 266 is sized such that a portion of cellular mass 270 is confined to lower portion 266.

Lower portion 266 may have a width less than 250 μm. In the case where cellular mass 270 is a denuded human zygote, the width may be less than 140 μm. In the case where cellular mass 270 is a denuded mammalian zygote, the width may be less than 70 μm. In the case where cellular mass 270 is a clump of mammalian cells, the width may be less than 50 μm. In the case where cellular mass 270 is a single mammalian cell, the width may be less than 5 μm.

FIG. 5b is an enlarged side view, partially broken-away and in cross-section, of funnel 322 and microchannel 330. Lower portion 366 is sized such that a portion of cellular mass 370 is confined to lower portion 366. Additionally, microchannel 330 is above lower portion 366.

FIG. 5c is an enlarged side view, partially broken-away and in cross-section, of funnel 422 and microchannel 430. Lower portion 466 and microchannel 430 are sized such that portions of cellular mass 470 may be in either of lower portion 466 and the portion of microchannel 430 adjacent lower portion 466.

Microfluidic devices may include a PDMS slab with bell-shaped microfluidic channel features, a culture media reservoir, and a funnel shaped well for culture. The media reservoir and funnel shaped well are connected with the microfluidic channels. The funnel shaped well may have an approach angle of approximately 60° to facilitate the one-step loading and unloading of cells and an approximately 500 μm diameter tip.

In funnel type wells, cells do not need to be moved to designated areas. Instead, cells loaded in the funnel remain stationary. The medium or chemical composition in the funnel can be gradually changed to mimic conditions cells experience in vivo. In addition, the dimensions of the channels connected to the funnel can be controlled through soft-lithography processes such that cells are confined to the funnel. Cells may then be subjected to diverse flow conditions.

PDMS slabs may be prepared by casting prepolymer (Sylgard 184, Dow-Corning) at a 1:10 curing agent-to-base ratio against positive relief features approximately 30 μm in height and 400 μm in width. The relief features may comprise SU-8 (MicroChem, Newton, Mass.) and be fabricated on a thin glass wafer, approximately 200 μm thick, using backside diffused-light photolithography.

Microfluidic devices may include a tapered well which at its tip has an opening which communicates with one or a plurality of microchannels. The well and microchannels may be filled with fluid. One or more cells, e.g., embryos, may be introduced into the well, for example, by pipet. The cells settle to the bottom, but are prevented from exiting the well due to them being larger than the microchannels.

Fluid may be introduced into the well continuously or discontinuously. The fluid may contain the necessary growth media for the cells. In a well with a single hole at the bottom, for example, fluid may be caused to rise in the well from the microchannels, introducing extra nutrients, and then to fall, removing fluid which now contains exogenous substances, e.g., waste, via the microchannels.

Introduction and removal of fluid can be made using conventional gravity pumps or constant flow gravity driven pumps. Introduction and removal of fluid can also be made by outside supplies, such as pumps, or by on-board or “semi-on-board” tactile actuator-based pumping systems.

Wells may have inlets at other locations and or heights rather than at the bottom, so long as the entrance ways are sized such that cells will not pass into the channels. For example, there may be an opening at the bottom of a well and an opening near the middle or top, with fluid being supplied at the bottom and being removed closer to the top.

Wells may have a polygonal shape whose walls are inclined, in either a linear or curved fashion, such that cells added to the well have a tendency to gravitate toward the bottom and center of the well.

The material in which a well is formed may be, for example, thermosetting resin, thermoplastic, metal, glass, or ceramic.

Microfluidic devices may include a top layer containing a well, and constructed of a relatively rigid material so as to provide support for elastomeric layers or layers of lesser strength or modulus below. The top layer may comprise a hard transparent material, such as glass or polymethylmethacrylate. The well may have a low surface roughness ranging, for example, between 5 μm Ra and 0.1 μm Ra.

The well may penetrate through the top layer, thus having an open, wide-mouthed end on one side of the top layer, and on the bottom layer, a relatively narrow hole which allows fluid communication with microchannels in the second layer.

The microchannels may be positioned closely with respect to the opening in the well to minimize misalignment. For example, misalignment should not exceed 50 μm. The second layer may also constitute the bottom layer, particularly when the microchannels are substantially on top of the second layer, e.g, abutting the bottom surface of the top layer.

Microfluidic devices may include microchannels that are, at least in part, along the bottom of the second layer. A third, or sealing layer may be applied thereto. This sealing layer may be rather thin, such that braille-type tactile actuators may act as valves and pumps for the various microchannels. By this means, for example, fluid can be caused to flow or to be pumped in one or both directions in a given microchannel depending upon the valving, whether the valves are on or off, and whether a pump is pumping one way or the other with respect to the microchannel.

In use, a well is first filled with fluid, e.g., an embryo culture medium, and one or more embryos added to the well. An oil overlay, produced by dropping one or two fine drops of oil onto the liquid surface in the well, is then provided.

The oil reduces or prevents evaporation of liquid from the well, thus stabilizing the osmolality, or concentration, of the ingredients therein. In the absence of such oil, the liquid may evaporate. The oil overlay also affects the flow of air, including specifically oxygen and CO₂ into the fluid, and the release of these gases from the fluid. The oil may be any compatible oil, for example, a silicone oil, a paraffin oil, or a polyethylene oligomer oil. For the same reason, the second or third layers, if present, may include, for example, parylene, or other materials, which minimize water loss.

The second and third layers may be made of cast elastomer, particularly when the embodiments employ tactile actuators. If “off-chip” fluid supply or valving is used, however, the use of an elastomer is not necessary, and other materials, such as cast epoxy, injection molded thermoplastic, or glass, can be used. The surface of these materials should be bio-compatible, and if not, should be coated appropriately.

Zygotes may be introduced into a well containing a fluid as is conventionally employed for embryo culture. The fluid in the well is then covered with oil and incubated at a suitable temperature. Fluid is directed into and out of the well through microchannels continuously or discontinuously, e.g., a back and forth type of fluid supply wherein the fluid level in the well increases and then decreases cyclically. The growing embryo may be inspected by conventional optical microscopy methods, and when judged grown to the proper stage, the embryo is removed from the well. If the top of the well is larger then the bottom, one-step removal is particularly easy and the risk of damage to the embryo is low.

Microfluidic devices may contain microchannels whose flow characteristics are to be actively varied and formed in a compressible or distortable elastomeric material such as an organopolysiloxane elastomer. Substrates, however, may be constructed of hard, e.g., substantially non-elastic material at portions where active control is not desired.

Microfluidic devices may contain at least one active portion which alters the shape or volume of chambers or passageways (“empty space”. Such active portions include mixing portions, pumping portions, valving portions, flow portions, channel or reservoir selection portions, cell crushing portions, and unclogging portions. These active portions induce some change in the fluid flow, fluid characteristics, channel, or reservoir characteristics by exerting a pressure on the relevant portions of the microfluidic device, and thus alter the shape or volume of the empty space which constitutes these features. The term “empty space” refers to the absence of substrate material. In use, the empty space may be filled with fluid.

The active portions may be activatable by pressure to close their respective channels or to restrict the cross-sectional area of the channels to accomplish the desired active control. To achieve this purpose, the channels or reservoirs may be constructed in such a way that modest pressure from the exterior of the microfluidic device causes the channels or reservoirs (“microfluidic features” to compress, causing local restriction or total closure of the respective feature.

Walls surrounding the feature and external surfaces may be elastomeric such that a minor amount of pressure causes an external surface and, optionally, the internal feature walls to distort, either reducing cross-sectional area at this point or completely closing the feature.

The pressure required to “activate” the active portion(s) of the device may be supplied by an external tactile device such as a refreshable Braille display. The tactile actuator contacts the active portion of the device, and when energized, extends and presses upon the deformable elastomer, restricting or closing the feature in the active portion.

Dimensions of the various flow channels and reservoirs may be determined by volume and flow rate properties. Channels which are designed for complete closure may be of a depth such that the elastomeric layer between the microchannel and the actuator can approach the bottom of the channel. Manufacturing the substrate of elastomeric material facilitates complete closure, in general, as does also a cross-section which is rounded, particularly at the furthest corners (further from the actuator). The depth will also depend, for example, on the extension possible for the actuator's extendable protrusions, e.g., pins. Thus, channel depths may vary, for example, from 1 nm to 500 μm.

Microfluidic devices may be prepared through the use of a negative photoresist, for example, SU-8 50 photoresist (Micro Chem Corp., Newton, Mass.) The photoresist may be applied to a glass substrate and exposed from the uncoated side through a suitable mask. Since the depth of cure is dependant on factors such as length of exposure and intensity of the light source, features ranging from very thin up to the depth of the photoresist may be created. The unexposed resist is removed, leaving a raised pattern on the glass substrate. The curable elastomer is cast onto this master and then removed.

The material properties of SU-8 photoresist and the diffuse light from an inexpensive light source can be employed to generate microstructures and channels with cross-sectional profiles that are rounded and smooth at the edges yet flat at the top, e.g, bell-shaped. Short exposures tend to produce a radiused top, while longer exposures tend to produce a flat top with rounded corners. Longer exposures also tend to produce wider channels. These profiles are ideal for use as compressive, deformation-based valves that require complete collapse of the channel structure to stop fluid flow. With such channels, Braille-type actuators produce full closure of the microchannels, thus producing a very useful valved microchannel. Such shapes also lend themselves to produce uniform flow fields, and have good optical properties as well.

In a typical procedure, a photoresist layer is exposed from the backside of the substrate through a mask, for example photoplotted film, by diffused light generated with an ultraviolet (UV) transilluminator. Bell-shaped cross-sections are generated due to the way in which the spherical wavefront created by diffused light penetrates into the negative photoresist. The exposure dose dependent change in the SU-8 absorption coefficient limits exposure depth at the edges.

The exact cross-sectional shapes and widths of the fabricated structures may be determined by a combination of photomask feature size, exposure time/intensity, resist thickness, and distance between the photomask and photoresist. Although backside exposure makes features which are wider than the size defined by the photomask and in some cases smaller in height compared to the thickness of the original photoresist coating, the change in dimensions of the transferred patterns is readily predicted from mask dimensions and exposure time.

The relationship between the width of the photomask patterns and the photoresist patterns obtained is essentially linear, e.g., slope of 1, beyond a certain photomask aperture size. This linear relationship allows straightforward compensation of the aperture size on the photomask through simple subtraction of a constant value. When exposure time is held constant, there is a threshold aperture size below which incomplete exposure will cause the microchannel height to be lower than the original photoresist thickness. Lower exposure doses will make channels with smoother and more rounded cross-sectional profiles. Light exposure doses that are too slow or photoresist thicknesses that are too large, however, are insufficient in penetrating through the photoresist, resulting in cross-sections that are thinner than the thickness of the original photoresist.

The suitability of bell-shaped cross-section microchannels of 30 μm thickness may be evaluated by exerting an external force onto the channel using a piezoelectric vertical actuator of commercially available refreshable Braille display. Spaces may be left between the membrane and the wall when the channel cross-section has discontinuous tangents, such as in rectangular cross-sections. In contrast, a channel with a bell-shaped cross-section may be fully closed under the same conditions. When a Braille pin is pushed against a bell-shaped or rectangular-shaped cross-section microchannel through a 200 μm PDMS membrane, the bell-shaped channels may be fully closed while the rectangular channels of the same width may have considerable leakage.

When used as deformation-based microfluidic valves, bell-shaped microchannels may show self-sealing upon compression compared to conventional rectangular or semi-circular cross-section channels. By way of example, a bell-shaped channel, having a width and height of 30 μm, may be completely closed by an 18 gf-force squeeze of a Braille pin.

Channels that have the bell-shaped cross-sections with gently sloping sidewalls may not be fabricated by melting resist technology, one of the most convenient methods to fabricate photomask-definable rounded patterns, because the profile is determined by surface tension.

Bell-shaped channels maximize the cross-sectional area within microfluidic channels without compromising the ability to completely close channels upon deformation. Furthermore, bell-shaped cross-sections provide channels with flat ceilings and floors, which is advantageous for reducing aberrations in optical microscopy and in obtaining flow fields with a more uniform velocity profile across the widths of the channel. These advantages of microchannels with bell-shaped cross-sectional shapes combined with the convenient, inexpensive, and commercially available valve actuation mechanism based on refreshable Braille displays will be useful for a wide range of microfluidic applications such as microfluidic cell culture and analysis systems, biosensors, and on-chip optical devices such as microlenses.

The extension outwards of tactile actuators should be sufficient for their desired purpose. Complete closure of a 40 μm deep microchannel, for example, will generally require a 40 μm extension, e.g., pin, or more when a single actuator is used, and about 20 μm or more when dual actuators on opposite sides of the channel are used.

For peristaltic pumping, mixing, and flow regulation, lesser extensions relative to channel height are useful. The areal size of the tactile activators may vary appropriately with channel width and function, and may range from 40 μm to about 2 mm. Larger and smaller sizes are possible as well.

A cellular mass, e.g., cells, may be cultured in microchannels and/or reservoirs within microfluidic devices. Additionally, cells may release various products, or active materials, e.g., immuno-modulators, metabolic substrates, nutrients, and growth factors, during cell culture. Unlike traditional cell culture devices, e.g., petri dishes, active materials released by a cell in a microfluidic cell culture device may noticeably affect the concentration of the media adjacent the cell. This is due, at least in part, to the relatively small volumes, e.g., 500 nanoliters, of media associated with microfluidic cell culture devices and the relatively lower diffusive transport of these active materials away from the media adjacent the cell, e.g., away from media within 450 microns of a surface of the cell. The above circumstances are also true in the presence of convection if convective transport of the active materials away from the fluid adjacent the cell is less than diffusive transport of the active materials away from the fluid adjacent the cell.

Cells typically prefer a range of concentrations for the active materials discussed above. In other words, there is a therapeutic window of concentration for a given active material and a given cell. Within the window, cell processes, e.g., growth, are optimized and the media thus provides the cell a therapeutic environment. In some circumstances, the therapeutic window may be a single value. In other circumstances, the therapeutic window may be a range of values.

Media prepared for use in non-microfluidic cell culture devices typically have initial concentrations of the materials discussed above falling within the respective material's therapeutic window. For example, if an optimum concentration of lactate in a media in a vicinity of a human embryo is 21.4 millimolars, a media prepared for use in a non-microfluidic cell culture device would have an initial concentration of lactate of at least 21.4 millimolars. That same media, however, may yield a sub-optimal concentration of lactate in a microfluidic cell culture device because the human embryo's net release of lactate would affect the concentration of the lactate in the media adjacent the embryo.

Microfluidic cell culture media for use with a cellular mass in a microfluidic cell culture device may be prepared taking into account that the cellular mass will affect the concentration of the products discussed above in the media adjacent the cellular mass. If an optimum concentration of lactate in a media in a vicinity of a human embryo is 21.4 milimolars, a media prepared for use in a microfluidic cell culture device may have an initial concentration of lactate less than 21.4 milimolars. This lower concentration may be found experimentally. For example, several cell culture media for use with a fertilized zygote from fertilization until the 8 cell stage may be prepared having initial lactate concentrations at 10, 15, 20, and 21.4 milimolars. Fertilized zygotes would be cultured at each concentration in a microfluidic cell culture device for 72 hours. At the expiration of the 72 hours, zygote development would be evaluated. If it is found that the media with an initial concentration of 20.0 millimolars of lactate was optimal for zygote development, a media for use with a microfluidic cell culture device would be prepared with an initial concentration of lactate of 20.0 millimolars.

A difference between the initial concentrations of lactate in media for use in a microfluidic cell culture device compared to a non-microfluidic cell culture device may indicate that the concentration of the product in the vicinity of a cell in the microfluidic device falls within the therapeutic window, e.g., that a zygote releases a net amount of lactate which causes the concentration of the lactate in the vicinity of the zygote to raise from an initial 20.0 millimolars to an optimum 21.4 millimolars.

Microfluidic cell culture media for use with microfluidic cell culture devices may be prepared for any cell. There are many ingredients in a media that are added to provide therapeutic benefit to cells which are also able to be provided by cells themselves. In such cases, media formulations where that ingredient is provided, in the base media, at concentrations lower than would be required for therapeutic benefits but still enable reaching the therapeutic level in the vicinity of cells can be provided. There are also additives that can be added to promote this self-production.

A variety of other cell-released molecules can be the subject of variations in concentrations, for example, immuno-modulators which may include soluble histocompatibility complexes, insoluble histocompatibility complexes, and interferons. Other examples include metabolic products which may include carbon dioxide, pyruvate, and carbohydrates, and nutrients which may include metabolites.

Additives, e.g., lipids, metabolic substrates, and growth factors, may be added to microfluidic cell culture media to further promote the endogenous production of some of the products discussed above by a cell. Examples of additives include metabolic substrates, e.g., glucose, pyruvate. Examples of lipid additives include Vitamin D3, dexamethasone, and glucocorticoid. Examples of metabolic substrate additives include pyruvate and carbohydrates.

More detailed examples of some of the metabolic molecules are described below.

Pyruvate is included as a substrate within the range of 0-5 mM for various culture media. Pyruvate is also a useful additive to enhance endogeneous production of carbon dioxide and its media equivalents.

Glucose is another example of a metabolic substrate. The amount of glucose in cell culture formulations range from 5.5 mM-55 mM. Many classical media are supplemented with 5.5 mM D-glucose (normal blood sugar levels in vivo). Several important media used as the base media for the design of proprietary media used in bio-manufacturing and tissue engineering contain diabetic levels (10 mM or over) of glucose supplementation. These levels of glucose require special formulation strategies to protect the cells and cell products from glucose mediated oxidative and carbonyl stress.

More detailed examples of some of the lipids are described below.

Linoleic acid is often included in the 200 nM-300 nM range in various media. Linoleic acid is a precursor to a number of other fatty acids (prostaglandins, prostacyclins, thromboxanes, phospho-lipids, glycolipids, and vitamins). These are important constituents of cell structures such as membranes, and long-term energy storage. Lipoic acid is included at 10 nM-1.0 uM in some basal media and many serum-free media. Lipoic acid is often required for the metabolism of pyruvate. Lipoic acid has been shown to overcome oxidative stress-induced insulin-resistance in vitro. Under conditions of oxidative stress, lipoic acid may replace insulin as an agent that supports increased glucose uptake. Lipoic acid regenerates endogenous antioxidants, removes transition metals from redox reactions by chelation, and reacts non-enzymatically (scavenging) with reactive oxygen species. Lipoic acid is able to increase glutathione (antioxidant) in cells. Lipoic acid helps protect cells from glutamate induced apoptosis by reducing extracellular L-cystine to L-cysteine. Lipoic acid can scavenge hydroxyl radicals; hypochlorous acid; peroxynitrous acid, and singlet oxygen.

More detailed examples of some of the hormones are described below.

Hydrocortisone(dexamethasone) is contained at 1-20 nM or 140-500 nM to increase plating efficiency, improve clonal growth, and stimulate the production of fibronectin. Insulin is contained at 0.1-10 ng/ml or 1-10 ug/ml in some media.

Some microfluidic cell culture devices may absorb a portion of the media and/or permit a portion of the media to evaporate during a cell culture period. This absorption and/or evaporation may cause shifts in the concentrations of certain products within the prepared media.

In general, the osmolality of the extracellular environment is normally ˜300 mmol/kg. Tolerance to higher osmolalities is cell type dependent. While Chinese Hamster Ovary (CHO) cells and a variety of hardy cell lines tolerate and proliferate under a wide range of osmolality (300-500 mmol/kg), more sensitive cells such as mammalian gametes and embryos will undergo a development that is blocked at osmolalities significantly lower or higher than 265 to 285 (mmol/kg).

If cells are placed in a solution of nonpenerating solutes having an osmolarity of 300 mmol/kg, they will neither swell nor shrink since the water concentrations in the intra- and extracellular fluid are the same, and the solutes cannot leave or enter. Such solutions are said to be isotonic, defined as having the same concentration of nonpenerating solutes as normal extracellular fluid. Solutions containing less than 300 mmol/kg of nonpenetrating solutes (hypotonic solutions) cause cells to swell because water diffuses into the cell from its higher concentration in the extracellular fluid. Solutions containing greater than 300 mmol/kg of nonpenetrating solutes (hypertonic solutions) cause cells to shrink as water diffuses out of the cell into the fluid with the lower water concentration. Therefore, it is important to maintain osmolarity levels during cell culture in vitro.

Microfluidic cell culture media for use with a microfludic cell device may be prepared taking into account that a certain amount of absorption and/or evaporation may occur. For example, if a solute is to have a concentration within a range to yield a therapeutic environment for the cell, the initial concentration of the solute in the prepared media would be lower than the range. The value of the concentration would be selected taking into account that a certain amount of the media will be absorbed and/or evaporated during the cell culturing event.

Evaporation may be detrimental to cell culture in microfluidic chips because the slight amount of evaporation from the small liquid volumes present in microfluidic systems may result in a significant increase in osmolality. Furthermore, microfluidic chips are especially prone to evaporation due to their fluidic compartments having very high surface areas for water to permeate or evaporate from relative to their volumes. It is well-documented that elevated osmolality can affect ion balance, cellular growth rate, metabolism, antibody production rate, signaling, and gene expression.

The solute may be a charged or neutral solute. Charged solutes may include at least one of sodium chloride, sodium bicarbonate, potassium chloride, and organic buffers. Charged solutes may include glutamine, glycine, and taurine. Charged solutes may include a protein. Neutral solutes may include carbohyrdrates such as glucose, trehalose, and galactose. Neutural solutes may include macromolecules such as polyethylene glycol, polyvinyl alcohol, dextran, polyvinylpyrrolidone, and polylysine.

Nonessential amino acids benefit cells through osmoregulation and cellular signaling. Most mammalian cells require mechanisms for regulatory volume increase or decrease in response to hormonal (e.g., insulin and glucagons) and other stimuli known to alter cellular volume. Additionally, preimplantation mouse embryos appear to develop in a somewhat hypertonic environment in oviductal fluid. In vitro, glycine, alanine, glutamine, and taurine protect preimplantation embryos from the otherwise detrimental effects of hypertonic media. For example, increased external osmolality causes an increase in intracellular glycine accumulation by early mouse embryos cultured from zygotes to two-cell embryos. Glycine functions as an intracellular osmolyte in embryos and the increase in total accumulated glycine at higher osmolality largely reflects increased intracellular concentration, since there is no significant difference between the volumes of two-cell embryos arising from culture in 240 vs. 310 mmol/kg. Intracellular accumulation of glycine seems to account for the osmoprotective effect of adding glycine to culture media.

The following examples describe media for use with microfluidic devices.

### EXAMPLE 1

In microfluidic channels, due to small dimensions and confined geometry, secreted molecules from a cell which are necessary for normal function will stay in the vicinity of the cell and increase the local concentration of that molecule. Because of this local increase in concentration, the concentration of the molecule in the original basal media can be lower than would be required in a macroscopic culture system where cell secreted molecules would rapidly diffuse away or be carried away by convection. Here, the control volume, or media adjacent the cells, is a volume within which a majority of the secreted molecules will be confined in during a given time period such as 600 seconds. The time period being defined by characteristics of the fluid refresh rate. Considering a microchannel with a cross section of 100 μm in width×100 μm in height and assuming one dimensional diffusion along the channel length, the diffusion length, L for lactate over the 600 second period is 1100 μm and the control volume will be 100 μm×100 μm×2200 μm=3*10⁻¹¹ m³=2.2*10⁻⁸ L. Here, lactate diffusion coefficient at 37° C. is 1.1×10⁻⁹ m²/s and diffusion length, L is defined as approximating diffusion to be mainly one-dimensional along the length of the channel. In contrast, in conventional static culture dishes, diffusion is three-dimensional with no confinement making the control volume approximately 10 times larger than in the microchannel described above.

Now a single bovine embryo in the blastocyst stage has a lactate production rate of 38.13 pmol/blasto/hr. Thus, 10 blastocysts will produce, within 600 seconds, approximately 6.36×10⁻¹¹ mol of lactate. In conventional G1/G2 media used for culture of bovine embryos, lactate concentration is 5.87 mmol/L in G2 media (G1 has 10 mmol/L). With the given control volume, the amount of lactate in conventional G2 media is 5.87×10⁻³×2.2*10⁻⁸=1.29×10⁻¹⁰ mol. Therefore, there would be an approximately 50% increase in the average lactate concentration in the control volume due to secreted lactate from embryos [(6.36×10⁻¹¹)/(1.29×10⁻¹⁰)×100=49.3%] if the conventional G2 media were used. In contrast, in culture dishes with a larger control volume, the local increase in average concentration would be only 5%. To account for this localization of embryo produced lactate within the particular microfluidic device described here, in the microfluidic optimized media, a reduced concentration of lactate (e.g. 2.5-3.5 mmol/L) is contained.

### EXAMPLE 2

This is a basal cell culture medium that enables long-term cell culture inside a microfluidic environment and outside of a CO2 incubator with long-term optical imaging. The medium has the following advantageous characteristics 1) high stability to light exposure, 2) limited but sufficient pH stability particularly in microfluidic cell cultures, 3) an amount of NaHCO3 that is below the conventional therapeutic window but is sufficient to satisfy immediate cellular needs for carbonates, and 4) additional additives that enhance endogenous production of carbon dioxide and its media equivalents.

More specifically, compared to conventional media, the buffers of this medium were reduced (10˜20 mM NaHCO3 and 5˜15 mM HEPES). To accommodate this reduction, fluctuations in pH were suppressed by using high concentrations of sodium pyruvate (2˜5 mM) to increase endogenous production of CO2; by addition of free base amino acids (1.5˜3 mM L-arginine; 0.7˜1.4 mM L-histidine; 0.5˜1 mM L-cysteine) which also enhance buffering capacity; and by using a combination of glucose (10˜20 mM) with galactose (25˜50 mM), rather than galactose alone, to help suppress pH drops due to rapid glycolysis. Because of the small-volume microfluidic cell culture, endogenously produced carbon dioxide and equivalents and other cell-generated products were retained in the cell vicinity to maintain concentrations of active products within a therapeautic window. In addition, the moderate riboflavin (0.2˜0.5:M), and phenol red (10˜20:M) concentrations used in the medium improves stability against light exposure.

In water, CO2 (carbon dioxide) spontaneously interconverts between CO2 (carbon dioxide) and H2CO3 (carbonic acid). The relative concentrations of CO2, H2CO3, and the deprotonated forms HCO3- (bicarbonate) and CO3-(carbonate) depend on pH.

The carbonate ion is a polyatomic anion with the empirical formula CO3- and is the conjugate base of bicarbonate, HCO3-1, which is the conjugate base of H2CO3 (carbonic acid).

NaHCO3 (sodium bicarbonate) is a salt consisting of the ions Na+ and the bicarbonate anion, HCO3-. The bicarbonate anion forms some hydroxide, which results in its solutions being mildly alkaline:
HCO3- →CO2+OH—

In this example, carbon dioxide and its media equivalents are the active material. Bicarbonates, carbonic acid, and carbonates are media equivalents of carbon dioxide.

### EXAMPLE 3

In conventional cultures, the local concentration of the active material decreases because of cellular consumption and because replenishing of the active material in the cell vicinity occurs mainly by diffusion only. To accommodate for this depletion and slow mass transport, the bulk media concentration of that material is set at a high concentration so that the decreased local concentration is still within the therapeautic window. In active microfluidic systems where there is efficient convective transport, the bulk concentration of the active material can be lowered because the material may be constantly refreshed around the cell.

### EXAMPLE 4

In many microfluidic manipulations, just the process of transferring small volumes of media from a large container to an on chip reservoir causes shifts in osmolality. For example, transferring 30 microliters of KSOM media from a bottle to a small shallow microreservoir over several minutes in a biological safety cabinet caused the osmolality to immediately shift upwards 15 mmol/kg. Thus in microfluidic media, the osmolality is decreased from 265 to 250 mmol/kg.

### EXAMPLE 5

To compensate for evaporation from the culture media, the osmolality of MCDB 131 microfluidic cell culture medium was adjusted to be lower than EGM2-MV, a proprietary media from Cambrex for human microvascular endothelial cells (HMVECs) with an osmolality range of 270-290 mmol/kg. Osmolality levels of MCDB 131 adjusted to be as low as 200 mmol/kg were experimentally tested to support proliferation and thus provide HMVECs with a therapeutic environment. Over a five day period, the rate of HMVEC proliferation supported by MCDB 131 with an osmolality of 200 mmol/kg was comparable to what is supported with EGM2-MV.

Evaporation during culture enables media initially with low osmolality to slightly increase their osmolality yet remain within a therapeutic range to support long-term proliferation. On the other hand, evaporation may result in the osmolality of culture media at normal levels to increase and leave its therapeutic osmolality range to support proliferation.

### EXAMPLE 6

Elevated osmolality can affect ion balance, cellular growth rate, metabolism, antibody production rate, signaling, and gene expression. In general, the osmolality of the extracellular environment is normally ˜300 mmol/kg. Tolerance to higher osmolalities is cell type dependent. More sensitive cells such as mammalian gametes and embryos will undergo a development that is blocked at osmolalities significantly lower or higher than 265 to 285 (mmol/kg).

Compared to conventional cell cultures performed in Petri dishes with low cell volume to extracellular fluid volume (CV/EV) ratios, microfluidic environments with large CV/EV ratios have many advantages in terms of cellular self-conditioning of their surrounding medium. Systems with large CV/EV ratios, however, typically also possess large surface to volume (SAV) ratios which increases the rate of evaporation and presents a challenge, particularly when using microfluidic devices made of water vapor permeable materials such as PDMS.

First, by evaporation occurring during the few minutes of handling between transfer of droplets from a macroscopic bottle of media to a well or microfluidic device and covering it with oil, osmolility will shift from 265 to 280 (mmol/kg) though media with 265 mmol/kg osmolality is prepared for culturing. So these losses of water should be compensated by lowering media osmolality by 15 mmol/kg for microfluidic cell culture.

Next, water permeation into microfluidic device over culture period will cause additional osmolality shifts in media, which will affect cellular functions or viability and necessitate a periodical refreshing time, t.

Using the simple diffusion equation,

$$\frac{Δ m}{A \cdot t} = D \cdot S \cdot {P}_{s} \cdot ( 1 - P / {P}_{s} ) \cdot \frac{1}{dx}$$

the amount of water loss, Δm in media or osmolality shifts over culture period under given humidity condition can be calculated.

Considering a PDMS microfluidic device which is composed of a bottom thin membrane for fluidic actuations and a thick (˜8 mm) PDMS slab with microfluidic channel feature with a cross section of 300 μm in width×30 μm in height and 50,000 μm in length forming a closed loop for recirculation, channel area, A is =1.5×10⁻⁵ m² and channel volume is 4.5×10⁻¹⁰m³. Here this microchannel is used for studying the effect of autocrine and paracrine effects on cells under fluid perfusion conditions over 24 hours and media need to be refreshed every 24 hours for each run. This refreshing time can be varied based on experiment's purpose.

Assuming that most evaporation over 24 hours occurs only through the thicker top PDMS (8 mm thickness) because bottom thin PDMS membrane is coated with parylene and that culture is conducted under a non-humidified environment (25%), the amount of water escaping through the top PDMS over 24 hours is 7.85×10⁻⁸ kg based on the simple diffusion equation. This amount of water loss causes osmolality shift. from 265 mmol/kg to 320 mmol/kg in given channel volume, 4.5×10⁻¹⁰m³(or 4.5×10⁻⁷ kg in water).

If the culture is conducted under a humidified incubator (85%), the amount of water escaping is 1.57×10⁻⁸ kg and the resulted osmolality shift is from 265 mmol/kg to 275 mmol/kg.

The increased amount by 10˜55 mmol/kg should be compensated by further lowering osmolality in media. Namely, to compensate total losses of water including loss during handling, lower osmolality media such as 195˜240 mmol/kg, which depends on humidity condition, is prepared for microfluidic cell culture.

Here approximate values of variables for the simple diffusion equation are: diffusion coefficient, D=3×10⁻⁹ m²/s, water solubility coefficient in PDMS,S=1.04 [(CM³)_{water}/cmHg·(cm³)_{PDMS}], saturated vapor pressure, P_{S} at 37° C.=6.33×10⁻³, P/Pₛ=0.25(25% humidity) or 0.85(85% humidity), dx=8000×10⁻⁶ (m). Here, the humidity at the media surface is considered to be 100% humidity at 37° C., and the saturated vapor specific volume (V_{g}) at 37° C. is V₈≈22.94(m³/kg), ρ_{g}=0.0436(kg/m³).

### EXAMPLE 7

Many media include scavengers to scavenge harmful products. Utilization of a microfluidic system incorporating dynamic flow of media can function as a scavenger of the waste produced by cells by washing the harmful products away. Thus the concentration of chemical or material scavenger can be reduced in a microfluidic media. Examples of harmful products include toxic metals and radicals. Examples of scavengers include ethylenediaminetetraacetic acid (EDTA), oil, thiols and ascorbate.

### EXAMPLE 8

Culture media optimized for traditional cell culture in macroscopic dishes in flasks when applied to microfluidic cell culture do not account for the high surface area-to-volume ratios present in microfluidic systems. The surface area-to-volume ratio is two to three orders of magnitude larger in microfluidic systems compared to macroscopic culture dishes and flasks. As a result, there is much more surface present in microfluidic systems compared to macroscopic culture flasks and dishes for proteins such as immunoglobulins, growth factors, cytokines, and other factors necessary to provide a therapeutic cellular environment to adsorb onto the surface and deplete the concentrations present in solution. Consequently, the high degree of non-specific adsorption of proteins and other factors in microfluidic cell culture systems may cause culture media that had been optimized to provide a therapeutic environment in macroscopic dishes and flasks to no longer provide a therapeutic environment when applied to microfluidic cell culture because the concentrations of the factors have dropped below a threshold level necessary to provide a therapeutic environment.

Optimization of media for microfluidic cell culture requires the amounts of factors necessary to remain above a threshold level to provide a therapeutic environment to be adjusted relative to the levels present in traditional cell culture media. For instance, in macroscopic endothelial cell culture, 50 ng/ml of vascular endothelial factor (VEGF) in culture media is added to provide the environment to stimulate endothelial cells to form vascular networks which is known to be a threshold response. Thus, in microfluidic culture conditions, the amount of VEGF added will need to be adjusted to account for increased protein adsorption and to ensure that endothelial cells form vascular networks.

Examples of instances when adsorption-mediated depletion of factors supporting a therapeutic environment is particularly high are: 1) long lengths (on the order of mm or cm) between the fluid inlet and the location of cells because this length of channel provides much surface for protein adsorption and 2) slow pumping conditions where diffusion effects dominate providing more time for proteins to adsorb.

Adsorption occurs more rapidly during initial exposure of device surfaces to media (starts absorbing in less than 1 seconds and will continue for up to hours depending on product adsorbing and concentrations.) For some products such as proteins, depletion by retention onto channel walls will greatly reduce after a monolayer of the product is formed on the walls.

Other products (other than proteins) that can be retained by device include lipids (steroids and some hormones and vitamins). Examples of proteins include albumin, globulins, growth factors (specifically VEGF and leukemia inhibitory factor (LIF) and fibroblast growth factor).

Appendix A discloses a handheld recirculation system and customized media for microfluidic cell culture. Appendix B discloses a device for embryo culture and use thereof. Appendix C discloses integrated microfluidic control employing programable tactile actuators. Appendix D discloses a computerized control method and system for microfluidics and computer program product for use therein. Embodiments of the invention may be used in the devices described in Appendices A, B, C, and D.

### Appendix A

Many modifications will be apparent to those skilled in the art, and are part of the subject matter disclosed herein. The clamping mechanism, for example, may be replaced or augmented by other clamping mechanisms, including simple clamps which are separate from but engageable with the fingerplate, or which can span the height of the entire device, including the braille display module.

In similar manner, while the transparent heating element is described as being fabricated on a glass slide, it will be appreciated that this glass slide may be incorporated into a disposable device, become an integrated part thereof rather than a separate device. While less favorable, the heating element may also be disposed directly on the microfluidics chip. The heating unit may also be patterned such that only portions of the glass slide or chip are heated, thus conserving electrical power as well as avoiding heat in areas where heating is not desired, for example in fluid storage areas.

In advanced versions of the present lab-on-chip, it is desirable to have a battery power supply, either one-time use or rechargeable, on the chip itself, together with electrical circuitry for controlled operation of the heater unit, and of the tactile actuators also, when this is desired. The ability to divorce the structure from corded power supplies allows the module to be easily transported to other stations for testing, analysis, etc., while preserving the microenvironment within the module.

The subject invention further pertains to PMDS or other elastomeric silicone structures which incorporate a film, coating, or membrane over all or only a portion of the module structure, which serves as a vapor barrier to minimize evaporation of liquids contained in the channels, reservoirs, etc., of the devices. Suitable vapor barriers are, in general, relatively pore free, hydrophobic films, e.g. of parylene. In addition, films which are resistant to the flow of oxygen, of carbon dioxide, or both these gases may also be applied to minimize any influence of the ambient atmosphere on the conditions established within the device. Such films are well known from the field of plastic, particularly polyethylene terephthalate, drink containers.

### Appendix B

It has now been surprisingly discovered that embryos may be grown with good survival rates in an efficient manner by growth at the bottom of a well which is in communication with a microchannel device supplying fluid to the well proximate its bottom. The bottom opening is sized so as not to allow the embryo to enter the channel.

The invention may be described with relation to the accompanying drawings, many of which illustrate the volumes or hollows, channels, etc. within the microfluidics device rather than the walls of the device themselves. As illustrated, the best mode of the device is a generally conical well which at its tip has an opening which communicates with one or a plurality of microchannels. The well is filled with fluid, as are the microchannels, and one or more embryos are introduced into the well, for example by pipet. The embryos settle to the bottom, but are prevented from exiting the well due to them being larger than the holes in the well.

Fluid may be introduced into the well continuously or discontinuously, the fluid preferably containing the necessary growth media for the embryo. For example, in a well with a single hole at the bottom, fluid may be caused to rise in the well from the microchannels, introducing extra nutrients, and then to fall, removing fluid which now contains exogenous substances (waste) via the microchannels.

Introduction and removal of fluid can be made using conventional gravity pumps, or constant flow gravity driven pumps. Fluid can also be supplied by outside supplies such as pumps, etc., or preferably by on-board or “semi-on board” tactile actuator-based pumping systems.

The well can also have inlets at other locations and or heights rather than exclusively at the bottom, so long as the entrance ways to the channels are sized such that the embryos will not pass into the channels. For example, there might be an opening at the bottom of the well and an opening near the middle or top, with fluid being supplied at the bottom, for example, and being removed closer to the top.

The well also need not be entirely conical in shape, but is preferably shaped such that the walls are inclined, regardless of whether linear or curved such that the embryo's will have a natural tendency to gravitate toward the bottom and center of the well. The material of the well is not overly critical, and may be thermosetting resin or thermoplastic, metal, glass, ceramic, etc. In preferred constructions, the device is a multilayer device, the top layer containing the well, and constructed of relatively rigid material so as to provide support for elastomeric layers or layers of lesser strength and/or modulus below.

Thus, it is preferable that the top layer be of hard transparent material such as glass, polymethylmethacrylate, etc. The conical well should have a low surface roughness, preferably below 5 μmm Ra, more preferably less than 1 μm Ra, and yet more preferably less than 0.1 μm Ra.

In preferred devices, the conical well penetrates entirely through the top layer, thus having an open, wide-mouthed end on one side of the top layer, and on the bottom this layer, a relatively narrow hole which allows fluid communication with the microchannels in the second layer. The second layer preferably directly abuts the first layer, and has one or a plurality of microchannels which are in fluid communication with the conical well. It is relatively important that the channels be positioned closely with respect to the opening in the well. For example misalignment should preferably be maximized at 50 μm. The second layer may also constitute the bottom layer, particularly when the microfluid channels are substantially on top of the second layer, i.e. abutting the bottom surface of the top layer. However, in preferred devices, the channels are at least in part along the bottom of the second layer and a third, or sealing layer is applied thereto. This sealing layer is preferably rather thin, such that braille-type tactile actuators may act as valves and pumps for the various microchannels. By this means, for example, fluid can be caused to flow or to be pumped in only one direction in a given microchannel, or can be bidirectional flow, depending upon the valving, whether the valves are on or off, and whether a pump is pumping one way or the other with respect to the channel.

In use, the device is first filled with fluid, for example an embryo culture medium, and one or more embryos added to the well, An oil overlay, produced by dropping one or two fine drops of oil onto the liquid surface in the well is then provided. The oil prevents evaporation of liquid from the well, thus changing the osmolality, or concentration, of the ingredients therein. It also affects the flow of air, including specifically oxygen and C0₂ into the fluid, and the release of these gases from the fluid. The oil may be any compatible oil, for example a silicone oil, a paraffin oil, a polyethylene oligomer oil, etc. For the same reason, portions of the apparatus in the second and/or third layers may be coated, for example with parylene or other coating which minimizes, particularly, water loss.

The second and third layers are preferably made of cast elastomer, particularly when the valving and pumping embodiments employing tactile actuators are employed. However, if “off-chip” fluid supply, valving, etc. is used, then use of an elastomer is not necessary, and other materials such as cast epoxy, injection molded thermoplastic, glass, etc., can be used. It is of course recognized that the surface of these materials should be compatible with embryo culture, and if not, should be coated appropriately.

The process of the subject invention requires introduction of zygote(s) into the well which contains fluid, preferably a growth fluid as is conventionally employed for embryo culture. The fluid in the conical well is then covered with oil, preferably mineral oil, and the device incubated at a suitable temperature. Fluid is directed into and out of the well through the microchannels continuously or discontinuously. For example, a back and forth type of fluid supply wherein the fluid level in the well increases and then decreases cyclically has been found most advantageous. The growing embryo may be inspected by conventional optical microscopy methods, and when judged grown to the proper stage, the embryo is removed from the well. Because the top of the well is larger then the bottom, removal is particularly easy and the risk of damage is low.

### Appendix C

The microfluidic devices of the present invention contain microchannels whose flow characteristics are to be actively varied, formed in a compressible or distortable elastomeric material. Thus, it is preferred that substantially the entire microfluidic device be constructed of a flexible elastomeric material such as an organopolysiloxane elastomer (“PDMS”, as described hereinafter. However, the device substrate may also be constructed of hard, i.e., substantially non-elastic material at portions where active control is not desired, although such construction generally involves added construction complexity and expense. The generally planar devices preferably contain a rigid support of glass, silica, rigid plastic, metal, etc. on one side of the device to provide adequate support, although in some devices, actuation from both major surfaces may require that these supports be absent, or be positioned remote to the elastomeric device itself.

The microfluidic devices of the present invention contain at least one active portion which alters the shape and/or volume of chambers or passageways (“empty space”, particularly fluid flow capabilities of the device. Such active portions include, without limitation, mixing portions, pumping portions, valving portions, flow portions, channel or reservoir selection portions, cell crushing portions, unclogging portions, etc. These active portions all induce some change in the fluid flow, fluid characteristics, channel or reservoir characteristics, etc. by exerting a pressure on the relevant portions of the device, and thus altering the shape and/or volume of the empty space which constitutes these features. The term “empty space” refers to the absence of substrate material. In use, the empty space is usually filled with fluids, microorganisms, etc.

The active portions of the device are activatable by pressure to close their respective channels or to restrict the cross-sectional area of the channels to accomplish the desired active control. To achieve this purpose, the channels, reservoirs, etc. are constructed in such a way that modest pressure from the exterior of the microfluidic device causes the channels, reservoirs, etc. (“microfluidic features” to compress, causing local restriction or total closure of the respective feature. To accomplish this result, the walls within the plane of the device surrounding the feature are preferably elastomeric, and the external surfaces (e.g., in a planar device, an outside major surface) are necessarily elastomeric, such that a minor amount of pressure causes the external surface and optionally the internal feature walls to distort, either reducing cross-sectional area at this point or completely closing the feature.

The pressure required to “activate” the active portion(s) of the device is supplied by an external tactile device such as are used in refreshable Braille displays. The tactile actuator contacts the active portion of the device, and when energized, extends and presses upon the deformable elastomer, restricting or closing the feature in the active portion.

Rather than close or restrict a feature by being energized, the tactile actuator may be manufactured in an extended position, which retracts upon energizing, or may be applied to the microfluidics device in an energized state, closing or restricting the passage, further opening the passage upon de-energizing.

The preferred actuators at the present time are programmable Braille display devices such as those previously commercially available from Telesensory as the Navigator™ Braille Display with Gateway™ software which directly translates screen text into Braille code. These devices generally consist of a linear array of “8-dot” cells, each cell and each cell “dot” of which is individually programmable. Such devices are used by the visually impaired to convert a row of text to Braille symbols, one row at a time, for example to “read” a textual message, book, etc. These devices are presently preferred because of their ready commercial availability. The microfluidic device active portions are designed such that they will be positionable below respective actutable “dots” or protrusions on the Braille display. Braille displays are available from Handy Tech, Blazie, and Alva, among other suppliers.

However, to increase flexibility, it is possible to provide a regular rectangular array usable with a plurality of microfluidics devices, for example having a 10×10, 16×16, 20×100, 100×100, or other array. The more close the spacing and the higher the number of programmable extendable protrusions, the greater is the flexibility in design of microdevices. Production of such devices follows the methods of construction known in the art. Addressability also follows from customary methods. Non-regular arrays, i.e. in patterns having actuators only where desired are also possible.

Suitable Braille display devices suitable for non-integral use are available from Handy Tech Electronik GmbH, Horb, Germany, as the Graphic Window Professional.TM. (GWP), having an array of 24×16 tactile pins. Pneumatic displays operated by microvalves have been disclosed by Orbital Research, Inc. said to reduce the cost of Braille tactile cells from 70 $ U.S. per cell to Ca. 5-10 $/cell. Piezoelectric actuators are also usable where a piezoelectric element replaces the electrorheological fluid, and electrode positioning is altered accordingly.

The microfluidic devices of the present invention have many uses. In cell growth, the nutrients supplied may need to be varied to simulate availability in living systems. By providing several supply channels with active portions to close or restrict the various channels, supply of nutrients and other fluids may be varied at will. An example is a three dimensional scaffolding system to create bony tissue, the scaffolding supplied by various nutrients from reservoirs, coupled with peristaltic pumping to simulate natural circulation.

A further application involves cell crushing. Cells may be crushed by transporting them in channels through active portions and actuating channel closure to crush the cells flowing through the channels. Cell detection may be achieved, for example, by flow cytometry techniques using transparent microfluidic devices and suitable detectors. Embedding optical fibers at various angles to the channel can facilitate detection and activation of the appropriate activators. Similar detection techniques, coupled with the use of valves to vary the delivery from a channel to respective different collection sites or reservoirs can be used to sort embryos and microorganisms, including bacteria, fungi, algae, yeast, viruses, sperm cells, etc.

Growth of embryos generally require a channel or growth chamber which is capable of accommodating the embryo and allowing for its subsequent growth. Such deep channels cannot effectively be closed, however. A microfluidics device capable of embryo growth may be fabricated by multiexposure photolithography, using two masks. First, a large, somewhat rectangular (200 μm width×200 μm depth) channel, optionally with a larger 200 μm deep by 300 μm length and 300 μm width growth chamber at one end is fabricated. Merging with the 200 μm×200 μm channel is a smaller channel with a depth of ca. 30 μm, easily capable of closure by a Braille pin. Exiting the bulbous growth chamber are one or more thin (30 μm) channels. In operation, embryo and media are introduced into the large channel and travel to the bulbous growth chamber. Because the exit channels from the growth chamber are very small, the embryo is trapped in the chamber. The merging channels and exit channels can be used to supply nutrients, etc., in any manner, i.e. continuous, pulsating, reverse flow, etc. The embryo may be studied by spectroscopic and/or microscopic methods, and may be removed by separating the elastomeric layer covering the PDMS body which houses the various channels.

Construction of fluidic devices is preferably performed by soft lithography techniques, as described, for example by D. C. Duffy et al., Rapid Prototyping of Microfluidic Systems in Poly(dimethylsiloxane), ANALYTICAL CHEMISTRY 70, 4974-4984 (1998). See also, J. R. Anderson et al., ANALYTICAL CHEMISTRY 72, 3158-64 (2000); and M. A. Unger et al., SCIENCE 288, 113-16 (2000). Addition-curable RTV-2 silicone elastomers such as SYLGARD.RTM. 184, Dow Corning Co., can be used for this purpose.

The dimensions of the various flow channels, reservoirs, growth chambers, etc. are easily determined by volume and flow rate properties, etc. Channels which are designed for complete closure must be of a depth such that the elastomeric layer between the microchannel and the actuator can approach the bottom of the channel. Manufacturing the substrate of elastomeric material facilitates complete closure, in general, as does also a cross-section which is rounded, particularly at the furthest corners (further from the actuator). The depth will also depend, for example, on the extension possible for the actuator's extendable protrusions. Thus, channel depths may vary quite a bit. A depth of less than 100 μm is preferred, more preferably less than 50 μm. Channel depths in the range of 10 μm to 40 μm are preferred for the majority of applications, but even very low channel depths, i.e. 1 nm are feasible, and depths of 500 μm are possible with suitable actuators, particularly if partial closure (“partial valving” is sufficient.

The substrate may be of one layer or a plurality of layers. The individual layers may be prepared by numerous techniques, including laser ablation, plasma etching, wet chemical methods, injection molding, press molding, etc. However, as indicated previously, casting from curable silicone is most preferred, particularly when optical properties are important. Generation of the negative mold can be made by numerous methods, all of which are well known to those skilled in the art. The silicone is then poured onto the mold, degassed if necessary, and allowed to cure. Adherence of multiple layers to each other may be accomplished by conventional techniques.

A preferred method of manufacture of some devices employs preparing a master through use of a negative photoresist. SU-8 50 photoresist from Micro Chem Corp., Newton, Mass., is preferred. The photoresist may be applied to a glass substrate and exposed from the uncoated side through a suitable mask. Since the depth of cure is dependant on factors such as length of exposure and intensity of the light source, features ranging from very thin up to the depth of the photoresist may be created. The unexposed resist is removed, leaving a raised pattern on the glass substrate. The curable elastomer is cast onto this master and then removed.

The material properties of SU-8 photoresist and the diffuse light from an inexpensive light source can be employed to generate microstructures and channels with cross-sectional profiles that are “rounded and smooth” at the edges yet flat at the top (i.e. bell-shaped). Short exposures tend to produce a radiused top, while longer exposures tend to produce a flat top with rounded corners. Longer exposures also tend to produce wider channels. These profiles are ideal for use as compressive, deformation-based valves that require complete collapse of the channel structure to stop fluid flow, as disclosed by M. A. Unger, et al., SCIENCE 2000, 288, 113. With such channels, Braille-type actuators produced full closure of the microchannels, thus producing a very useful valved microchannel. Such shapes also lend themselves to produce uniform flow fields, and have good optical properties as well.

In a typical procedure, a photoresist layer is exposed from the backside of the substrate through a mask, for example photoplotted film, by diffused light generated with an ultraviolet (UV) transilluminator. Bell-shaped cross-sections are generated due to the way in which the spherical wavefront created by diffused light penetrates into the negative photoresist. The exposure dose dependent change in the SU-8 absorption coefficient (3985 m⁻¹ unexposed to 9700 m⁻¹ exposed at 365 nm) limits exposure depth at the edges.

The exact cross-sectional shapes and widths of the fabricated structures are determined by a combination of photomask feature size, exposure time/intensity, resist thickness, and distance between the photomask and photoresist. Although backside exposure makes features which are wider than the size defined by the photomask and in some cases smaller in height compared to the thickness of the original photoresist coating, the change in dimensions of the transferred patterns is readily predicted from mask dimensions and exposure time. The relationship between the width of the photomask patterns and the photoresist patterns obtained is essentially linear (slope of 1) beyond a certain photomask aperture size. This linear relationship allows straightforward compensation of the aperture size on the photomask through simple subtraction of a constant value. When exposure time is held constant, there is a threshold aperture size below which incomplete exposure will cause the microchannel height to be lower than the original photoresist thickness. Lower exposure doses will make channels with smoother and more rounded cross-sectional profiles. Light exposure doses that are too slow (or photoresist thicknesses that are too large), however, are insufficient in penetrating through the photoresist, resulting in cross-sections that are thinner than the thickness of the original photoresist.

The suitability of bell-shaped cross-section microchannels of 30 μm thickness to be used as deformation-based valves was evaluated by exerting an external force onto the channel using a piezoelectric vertical actuator of commercially available refreshable Braille displays. Spaces may be left between the membrane and the wall when the channel cross-section has discontinuous tangents, such as in rectangular cross-sections. In contrast, a channel with a bell-shaped cross-section is fully closed under the same conditions. When a Braille pin is pushed against a bell-shaped or rectangular-shaped cross-section microchannel through a 200 μm poly(dimethylsiloxane) (PDMS) membrane, the bell-shaped channels were fully closed while the rectangular channels of the same width had considerable leakage.

The technique described is cost- and time-effective compared to other photolithographic methods for generating well defined rounded profiles such as gray-scale mask lithography, or laser beam polymerization because there is no need for special equipment such as lasers, collimated light sources (mask aligner), or submicron resolution photomasks; it only requires a transilluminator available in many biological labs. In addition, the backside exposure technique can generate more profiles compared to other soft lithography-based patterning methods such as microfluidic mask lithography and the use of patterned laminar flows of etchant in an existing microchannel.

When used as deformation-based microfluidic valves, these bell-shaped microchannels showed improved self-sealing upon compression compared to conventional rectangular or semi-circular cross-section channels as demonstrated by simulations, and by experiments. A bell-shaped channel (width: 30 μm; height 30 μm) was completely closed by an 18 gf-force squeeze of a Braille pin. It is notable that channels that have the bell-shaped cross-sections with “gently sloping” sidewalls cannot be fabricated by melting resist technology, one of the most convenient methods to fabricate photomask-definable rounded patterns, because the profile is determined by surface tension. The bell-shaped channels maximize the cross-sectional area within microfluidic channels without compromising the ability to completely close channels upon deformation. For example, the channel cross-section described here is larger than previously reported, pneumatically actuated deformation-based valves (100 μm in width; 20 μm in height) and may be more suitable for mammalian cell culture. Furthermore, the bell-shaped cross-sections provide channels with flat ceilings and floors, which is advantageous for reducing aberrations in optical microscopy and in obtaining flow fields with a more uniform velocity profile across the widths of the channel. These advantages of microchannels with bell-shaped cross-sectional shapes combined with the convenient, inexpensive, and commercially available valve actuation mechanism based on refreshable Braille displays will be useful for a wide range of microfluidic applications such as microfluidic cell culture and analysis systems, biosensors, and on-chip optical devices such as microlenses.

The extension outwards of the tactile actuators must be sufficient for their desired purpose. Complete closure of a 40 μm deep microchannel, for example, will generally require a 40 μm extension (“protrusion” or more when a single actuator is used, and about 20 μm or more when dual actuators on opposite sides of the channel are used. For peristaltic pumping, mixing, and flow regulation, lesser extensions relative to channel height are useful. The areal size of the tactile activators may vary appropriately with channel width and function (closure, flow regulation, pumping, etc.), and may preferably range from 40 μm to about 2 mm, more preferably 0.5 mm to 1.5 mm. Larger and smaller sizes are possible as well. The actuators must generate sufficient force. The force generated by one Braille-type display pin is approximately 176 mN, and in other displays may be higher or lower.

By use of the present invention, numerous functions can be implemented on a single device. Use of multiple reservoirs for supply of nutrients, growth factors, etc. is possible. The various reservoirs make possible any combination of fluid supply, i.e. from a single reservoir at a time, or from any combination of reservoirs. This is accomplished by establishing fluid communication with a reservoir by means of a valved microchannel, as previously described. By programming the Braille display or actuator array, each individual reservoir may be connected with a growth channel or chamber at will. By also incorporating a plurality of extendable protrusions along a microchannel supply, peristaltic pumping may be performed at a variety of flow rates. Uneven, pulsed flow typical of vertebrate circulatory systems can easily be created. Despite the flexibility which the inventive system offers, construction is straightforward. The simplicity of the microfluidics device per se, coupled with a simple, programmable external actuator, enables a cost-effective system to be prepared, where the microfluidic device is relatively inexpensive and disposable, despite its technological capabilities.

Combinatorial, regulated flow with multiple pumps and valves that offer more flexibility in microfluidic cell studies in a laptop to handheld-sized system are created by using a grid of tiny actuators on refreshable Braille displays. These displays are typically used by the visually impaired as tactile analogs to computer monitors. Displays usually contain 20-80 rows of cells, each holding 8 (4×2) vertically moving pins (˜1-1.3 mm). Two pins on the same cell may typically be 2.45 mm apart center to center and 3.8 mm apart on different cells. Each pin may have the potential to protrude 0.7˜1 mm upward using piezoelectric mechanisms, and may hold up to ˜15-20 cN. Control of Braille pins actuators is accomplished by changing a line of text in a computer program. Unique combinations of Braille pins will protrude depending on the letters displayed at a given time. Braille displays are pre-packaged with software, easy to use, and readily accessible. They are designed for individual use, and range from walkman to laptop sizes while using AC or battery power. By using the moving Braille pins against channels in elastomeric, transparent rubber, it is possible to deform channels and create in situ pumps and valves.

### Appendix D

Embodiments of microfluidic devices may be suitable for the culture of a living organism in a fluid. A microfluidic device may control the flow and composition of fluids provided to the living organism. The microfluidic device may provide laminar, pseudo-multiple laminar or non-laminar flows. The microfluidic device may perform physical operations on the living organism. The microfluidic device may be used, for example, for general cell culture including cell washing and detachment, cell seeding and culture. The microfluidic device may be used as a microreactor, a tissue culture device, a cell culture device, a cell sorting device, a cell crushing device, a micro flow cytometer, a motile sperm sorter, a micro carburetor, a micro spectrophotometer, or a microscale tissue engineering device. The microfluidic device may includes sensors to determine states or flow characteristics of elements of the microfluidic device or the passage of particles in a channel. The sensors may be, for example, optical, electrical, or electromechanical sensors.

In one embodiment, a microfluidic device includes microchannels having flow characteristics that are actively varied and formed in a compressible or distortable elastomeric material. In one embodiment, the entire microfluidic device is constructed of a flexible elastomeric material, such as an organopolysiloxane elastomer (“PDMS”, as described hereinafter. However, the device substrate may also be constructed of hard, e.g., substantially non-elastic material at portions, where active control is not desired.

The microfluidic devices may contain at least one active portion that alters the shape and/or volume of chambers or passageways (“empty space”, particularly fluid flow capabilities of the device. Such active portions include, without limitation, mixing portions, pumping portions, valving portions, flow portions, channel or reservoir selection portions, cell crushing portions, and unclogging portions. These active portions all induce some change in the fluid flow, fluid characteristics, channel or reservoir characteristics, by exerting a pressure on the relevant portions of the device, and thus altering the shape and/or volume of the empty space which constitutes these features. The term “empty space” refers to the absence of substrate material. In use, the empty space is usually filled with fluids or microorganisms.

The active portions of the device are activatable by pressure to close their respective channels or to restrict the cross-sectional area of the channels to accomplish the desired active control. To achieve this purpose, the channels, reservoirs, or other elements are constructed in such a way that modest pressure from the exterior of the microfluidic device causes the channels, reservoirs or other elements (“microfluidic features” to compress, causing local restriction or total closure of the respective feature. To accomplish this result, the walls within the plane of the device surrounding the feature are preferably elastomeric, and the external surfaces (e.g., in a planar device, an outside major surface) are elastomeric, such that a minor amount of pressure causes the external surface and optionally the internal feature walls to distort, either reducing cross-sectional area at this point or completely closing the feature.

The pressure used to “activate” the active portion(s) of the device is supplied by an external tactile device, such as are used in refreshable Braille displays of the actuator system. The tactile actuator contacts the active portion of the device, and when energized, extends and presses upon the deformable elastomer, restricting or closing the feature in the active portion.

In some embodiments, rather than close or restrict a feature by being energized, the tactile actuator may be manufactured in an extended position, which retracts upon energizing, or may be applied to the microfluidic device in an energized state, closing or restricting the passage, further opening the passage upon de-energizing.

A significant improvement in the performance, not only of the subject invention devices, but of other microfluidic devices which use pressure, e.g., pneumatic pressure, to activate device features, may be achieved by molding the device to include one or more voids adjacent the channel walls. These voids allow for more complete closure or distortion of the respective feature.

In one embodiment, the actuator system is a programmable Braille display that includes a plurality of moveable pins that each engage a corresponding element of the microfluidic device to perform a fluidic operation. The elements of the microfluidic device include pumps and valves. The pins may be arranged in a regular geometric array. Such arrangement maybe used with different configurations of the microfluidic device. In this arrangement, some pins may not be used for particular microfluidic devices because no element in the device corresponds to the pin. Alternatively the pins may be selected to correspond to elements of a specific or a group of multifluidic devices. Each pin may be controlled independently, and individually addressable.

An example of an actuator system is a Telesensory system such as the Navigator™ Braille Display with Gateway™ software, which directly translates screen text into Braille code. These devices generally comprise a linear array of “8-dot” cells, each cell and each cell “dot” of which is individually programmable. Such devices are used by the visually impaired to convert a row of text to Braille symbols, one row at a time, for example to “read” a textual message or book. The microfluidic device active portions are designed such that they will be positionable below respective actuable “dots” or protrusions on the Braille display. Braille displays are available from Handy Tech, Blazie, and Alva, among other suppliers. As will be described below, the system may use various software programs for controlling the pins of the actuator system by allowing the user to select processes to be performed on the organism, and then executing processes from a library.

However, to increase flexibility, it is possible to provide a regular rectangular array usable with a plurality of microfluidic devices, for example having a 10×10, 16×16, 20×100, 100×100, or other size array. The closer the spacing and the higher the number of programmable extendable protrusions, the greater is the flexibility in design of microdevices. Production of such devices follows the methods of construction known in the art. Addressability also follows from customary methods. Non-regular arrays, e.g., in patterns having actuators only where desired are also possible.

Devices can also be constructed which integrate the tactile actuators with the microfluidic device. The actuators are still located external to the microfluidic device itself, but attached or bonded thereto to form an integrated whole. Other types of actuator systems may be used, such as a tactile actuator device, which employs a buildup of an electrorheological fluid, an electromechanical Braille-type device employing shape memory wires for displacement between “on” and “off” portions, devices employing electrorheologic or magnetorheologic working fluids or gels, a pneumatically operated Braille device, “voice coil” type structures, especially those employing strong permanent magnets, devices employing shape memory alloys and intrinsically conducting polymer sheets.

Suitable Braille display devices suitable for non-integral use are available from Handy Tech Electronik GmbH, Horb, Germany, as the Graphic Window Professional™ (GWP), having an array of 24×16 tactile pins. Piezoelectric actuators are also usable where a piezoelectric element replaces the electrorheological fluid, and electrode positioning is altered accordingly.

The microfluidic device has many uses. The software described herein automates the operation of these uses. In cell growth, the nutrients supplied may be varied to simulate availability in living systems. By providing several supply channels with active portions to close or restrict the various channels, supply of nutrients and other fluids may be varied at will. An example is a three dimensional scaffolding system to create bony tissue, the scaffolding supplied by various nutrients from reservoirs, coupled with peristaltic pumping to simulate natural circulation.

Another application involves cell crushing. Cells may be crushed by transporting them in channels through active portions and actuating channel closure to crush the cells flowing through the channels. Cell detection may be achieved, for example, by flow cytometry techniques using transparent microfluidic devices and suitable detectors. Embedding optical fibers at various angles to the channel can facilitate detection and activation of the appropriate activators. Similar detection techniques, coupled with the use of valves to vary the delivery from a channel to respective different collection sites or reservoirs can be used to sort embryos and microorganisms, including bacteria, fungi, algae, yeast, viruses, and sperm cells.

The software controls the actuator system to control the pressure and thus the opening and closing of the channel and the timing. Depending on the processes to be performed, the software may address the actuators individually or in groups, and in patterns to provide actions, such as a peristaltic pumping action or a mixing action with respect to fluid in the channel. The software may monitor the sensors of the microfluidic device to selectively control the channel flow.

As an illustrative example of peristaltic pump formed by three pins engaging the microfluidic device, a pattern, such as XXO, OXX, OOX, XOX in repetition, where X is a closed position and O is an open position, to pump fluid in a channel may be used. The resultant fluid flow is pulsatile, with transient movements in both directions. The net movement can be predicted by its linear relationship to the pattern change frequency, and flow direction can be switched by reversing the pattern of actuation.

By use of the present invention, numerous functions can be implemented on a single device. Use of multiple reservoirs for supply of nutrients, growth factors, and the like is possible. The various reservoirs make possible any combination of fluid supply, e.g., from a single reservoir at a time, or from any combination of reservoirs. This is accomplished by establishing fluid communication with a reservoir by means of a valved microchannel, as previously described. By programming the actuator system, each individual reservoir may be connected with a growth channel or chamber at will. By also incorporating a plurality of extendable protrusions along a microchannel supply, peristaltic pumping may be performed at a variety of flow rates. Uneven, pulsed flow typical of vertebrate circulatory systems can easily be created. Combinatorial, regulated flow with multiple pumps and valves that offer more flexibility in microfluidic cell studies are created by using a grid of tiny actuators on refreshable Braille displays and executed automatically by software in response to user selections of processes to be performed.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A microfluidic cell culture media for a cellular mass in a microfluidic cell culture device, the media providing the cellular mass a therapeutic environment, the cellular mass having an active material, the media comprising:
a fluid having an initial concentration of the active material, the active material of the fluid being capable of being used by the cellular mass for at least a portion of one cell process, the fluid being capable of receiving active material released by the cellular mass, the initial concentration being less than a therapeutic window of concentration necessary to provide the cellular mass a therapeutic environment.

2. The media of claim 1 wherein the initial concentration is less than a therapeutic window of concentration necessary to provide the cellular mass a therapeutic environment if convective transport of the active material away from the fluid adjacent the cellular mass is less than diffusive transport of the active material away from the fluid adjacent the cellular mass.

3. The media of claim 1 wherein the fluid, if adjacent the cellular mass, has a concentration of the active material falling within the therapeutic window of concentration.

4. The media of claim 1 further comprising an additive that promotes endogenous production of the active material by the cellular mass.

5. The media of claim 4 wherein the additive is a lipid.

6. The media of claim 5 wherein the lipid comprises at least one of Vitamin D3, dexamethasone, and glucocorticoid.

7. The media of claim 4 wherein the additive is a metabolic substrate.

8. The media of claim 7 wherein the metabolic substrate is pyruvate.

9. The media of claim 7 wherein the metabolic substrate is a carbohydrate.

10. The media of claim 4 wherein the additive is a growth factor.

11. The media of claim 1 wherein the active material is an immuno-modulator.

12. The media of claim 11 wherein the immuno-modulator comprises at least one of a soluble histocompatibility complex, an insoluble histocompatibility complex, and an interferon.

13. The media of claim 1 wherein the active material is a metabolic active material.

14. The media of claim 13 wherein the metabolic active material is carbon dioxide.

15. The media of claim 13 wherein the metabolic active material is pyruvate.

16. The media of claim 13 wherein the metabolic active material is a carbohydrate.

17. The media of claim 1 wherein the active material is a nutrient.

18. The media of claim 17 wherein the nutrient is a metabolite.

19. The media of claim 1 wherein the active material is a growth factor.

20. A microfluidic cell culture media for use, during a cell culture period, with a cellular mass in a microfluidic cell culture device, the device being configured to at least one of absorb a portion of the media and permit a portion of the media to evaporate, the media comprising:
a water-based fluid having an initial concentration of an active material less than a therapeutic window of concentration necessary to provide the cellular mass a therapeutic environment during the cell culture period, the initial concentration having a value such that if a predetermined amount of water from the fluid is at least one of absorbed and evaporated during the cell culture period, the concentration of the active material in the fluid adjacent the cellular mass will fall within the therapeutic window of concentration.

21. The media of claim 20 wherein the active material is a solute.

22. The media of claim 21 wherein the solute is a charged solute.

23. The media of claim 22 wherein the charged solute comprises a salt.

24. The media of claim 23 wherein the salt comprises at least one of sodium chloride, sodium bicarbonate, potassium chloride, and an organic buffer.

25. The media of claim 23 wherein the charged solute comprises an amino acid.

26. The media of claim 25 wherein the amino acid is one of glutamine, glycine, and taurine.

27. The media of claim 22 wherein the charged solute is a macromolecule.

28. The media of claim 27 wherein the macromolecule is a protein.

29. The media of claim 21 wherein the solute is a neutral solute.

30. The media of claim 29 wherein the neutral solute comprises a carbohydrate.

31. The media of claim 30 wherein the carbohydrate is one of glucose, trehalose, and galactose.

32. The media of claim 29 wherein the neutral solute comprises a macromolecule.

33. The media of claim 32 wherein the macromolecule is one of polyethylene glycol, polyvinyl alcohol, dextran, polyvinylpyrrolidone, and polylysine.

34. A microfluidic cell culture media for use, during a cell culture period, with a cellular mass in a microfluidic cell culture device, the device being configured to at least one of absorb a portion of the media and permit a portion of the media to evaporate, the media comprising:
a water-based fluid having an initial osmolality less than a therapeutic window of osmolality necessary to provide the cellular mass a therapeutic environment during the cell culture period, the initial osmolality having a value such that if a predetermined amount of water from the fluid is at least one of absorbed and evaporated during the cell culture period, the osmolality of the fluid adjacent the cellular mass will fall within the therapeutic window of osmolality.
